(19) United States
(12) Patent Application Publication
(10) Pub. No.: US 2009/0299437 A1
(43) Pub. Date: Dec. 3, 2009

(54) Conductive Coating of Implants with Inductive Link

(75) Inventor: Martin Zimmerling, Patsch (AT)

Correspondence Address: Sunstein Kann Murphy & Timbers LLP, 125 SUMMER STREET, BOSTON, MA, 02110-1618, US

(73) Assignee: MED-EL Elektromedizinische Geraete GmbH, Fuerstenweg (AT)

(21) Appl. No.: 12/476,704

(22) Filed: Jun. 2, 2009

Related U.S. Application Data
(60) Provisional application No. 61/058,319, filed on Jun. 3, 2008.

(51) Int. Cl. A61F 11/04; A61N 1/36

(52) U.S. Cl. 607/57

(57) ABSTRACT

An implantable device includes an implanted coil for receiving a transcutaneous coil signal from an external transmitting coil. A coil housing contains the coil and has a non-conductive surface. A conductive coating covers at least a portion of the housing surface and forms a non-shielding pattern that minimizes interaction with the coil signal.

This application claims priority from U.S. Provisional Patent Application 61/058,319, filed Jun. 3, 2008, which is incorporated herein by reference.

### FIELD OF THE INVENTION

The present invention relates to medical implants, and more specifically to a surface coating for such devices.

### BACKGROUND ART

Some implantable devices such as Cochlear Implants (CI's) transfer electrical energy and data via an inductive link through the skin. This requires that the implanted receiving coils are not electrically shielded, which would interfere with the signal transfer. For that reason, implant coils are either encapsulated by a non-metallic housing (e.g. made of ceramics) or are embedded into silicone outside the hermetic encapsulation of the electronic circuit.

Just as with any surgical procedure, there is also some risk during implant surgery of postoperative infections at the surgical site. This risk is generally small and depends on several factors including hygiene standards in the operating room and surgical technique. One technical solution to further reduce the risk of bio-film growth and infection at the implant device is an antibiotic coating. One specific example would be a silver-based coating since silver ions are antibiotic (even against drug-resistant bacteria) and also prevent fungal decay around the implanted device. Depending on several factors (such as the silver concentration) a problem may arise in that silver coating over the inductive coil may cause some electrical shielding of the inductive link, thereby negatively affecting both power transfer to the implant device and also data communication in both directions. The inductive link may be influenced even if there is a high DC resistance of the conductive coating.

Implant devices also may have an internal magnet in the center of the implanted coil for providing an attractive magnetic force to a corresponding external magnet in the external coil. In some designs the internal magnet may be removable such as for magnetic resonance imaging (MRI) in order to avoid interactions between the internal magnet and the external MRI magnetic fields the attendant potential risks such as torque on the implant device, imaging artifacts and weakening of the internal magnet. A typical procedure is a first surgery to remove the internal magnet or to replace the magnet by a non-metallic space holder prior to MRI scanning, and then after the MRI scanning, a second surgery to replace the internal magnet.

Depending on the design of the removable magnet, there may be some dead space between the internal magnet and the surrounding part of the implant (e.g. a silicone material containing the implant coil). Such a dead space can potentially raise a risk of bio-film formation and associated infection which is difficult to treat.

Currently, various ways to avoid some of these problems include:

No conductive coating in the area of the inductive coil

Keep the conductive coating at a low level where the inductive link is not negatively affected

For the internal magnet, to have no removable magnet or have a design (geometry) which keeps the dead space very small.

### SUMMARY OF THE INVENTION

Embodiments of the present invention are direct to an implantable device that includes an implanted coil for receiving a transcutaneous coil signal from an external transmitting coil. A coil housing contains the coil and has a non-conductive surface. A conductive coating covers at least a portion of the housing surface and forms a non-shielding pattern that minimizes interaction with the coil signal.

In further specific embodiments, to claim 1, wherein the non-shielding pattern may form a web, mesh, and/or radial line pattern. The conductive coating may be an antibiotic coating and/or a silver-based coating.

The coil housing may be formed of a ceramic material and may also contain a signal processing module for processing the received coil signal. Embodiments may also have an electrode lead connected to the coil housing, wherein the conductive coating pattern further covers at least a portion of the electrode lead. The implantable device may be an element in a cochlear implant system.

Embodiments of the present invention also include an implantable device including an implanted magnet that interacts with an external magnet to maintain the external magnet in a constant position adjacent to the implanted magnet. A magnet housing contains the magnet. A therapeutic coating is between at least a portion of the magnet and the magnet housing for delivery of a therapeutic benefit in the vicinity of the therapeutic coating.

In further such embodiments, the therapeutic coating may specifically be an antibiotic coating and the therapeutic benefit may include an antibiotic effect. The therapeutic coating may be a silver-based coating and/or a colloidal-based coating, and the therapeutic benefit may include preventing formation of a bio-film in the vicinity of the therapeutic coating.

The implanted magnet may be a removable magnet. The magnet housing may be formed of a ceramic material and/or may further contain an implanted coil for receiving a transcutaneous coil signal from an external transmitting coil. The magnet housing also may include a signal processing module for processing the received coil signal. The implantable device may be an element in a cochlear implant system.

Embodiments of the present invention also include an implantable device having an implanted coil for receiving a transcutaneous coil signal from an external transmitting coil. A coil housing contains the implanted coil which is embedded in a non-shielding pattern of conductive containment material divided by non-conductive separating structures, and the pattern minimizes interaction of the containment material with the coil signal.

In specific such embodiments, the non-shielding pattern may form a web, mesh, or radial line pattern. The containment material may include an antibiotic component and/or a silver-based component and/or a colloidal-based component. The coil housing may be formed of a ceramic material and/or may also contain a signal processing module for processing the received coil signal. The implantable device may be an element in a cochlear implant system.

### BRIEF DESCRIPTION OF THE DRAWINGS

FIG. 1 shows an implantable device having a patterned conductive coating according to an embodiment of the present invention.

FIG. 2 shows another type of implantable device having a patterned conductive coating according to another embodiment of the present invention.

FIG. 3 shows an implantable device having inductive link coils embedded in a low conductivity structure according to an embodiment of the present invention.

FIG. 4 A-B shows an implantable device having a removable magnet and using a therapeutic coating according to an embodiment of the present invention.

FIG. 5 A-B shows another implantable device having a removable magnet and using a therapeutic coating according to another embodiment of the present invention.

### DETAILED DESCRIPTION OF SPECIFIC EMBODIMENTS

Embodiments of the present invention are directed to an implantable device that uses a surface coating and/or bulk material which are developed in a pattern that avoids many of the problems that arise in previous approaches. Some of the benefits which specific embodiments of a therapeutic surface coating may provide include, without limitation:
unimpeded data and energy transfer through the inductively coupled transcutaneous link
avoidance of RF-heating of the surface coating due to eddy currents (e.g. in the event of Magnetic Resonance Imaging (MRI) or even during normal use).
   This may be especially important during the charging phase of an implanted battery when a relatively high amount of RF power is sent over the inductive link.
good back-telemetry data transfer properties.

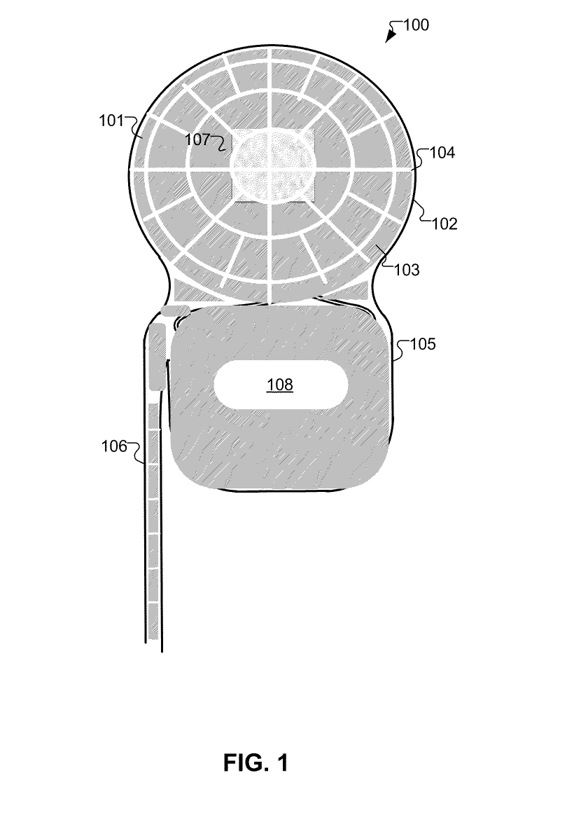

FIG. 1 shows an implantable device 100 having a patterned conductive coating 101 according to an embodiment of the present invention. The upper circular portion is a coil housing 102 containing an implanted coil 103 for receiving a transcutaneous coil signal from an external transmitting coil. The coil housing 102 also contains an internal magnet 107 for maintaining an external magnet of an external transmitting coil in a constant position adjacent to the implanted magnet 107.

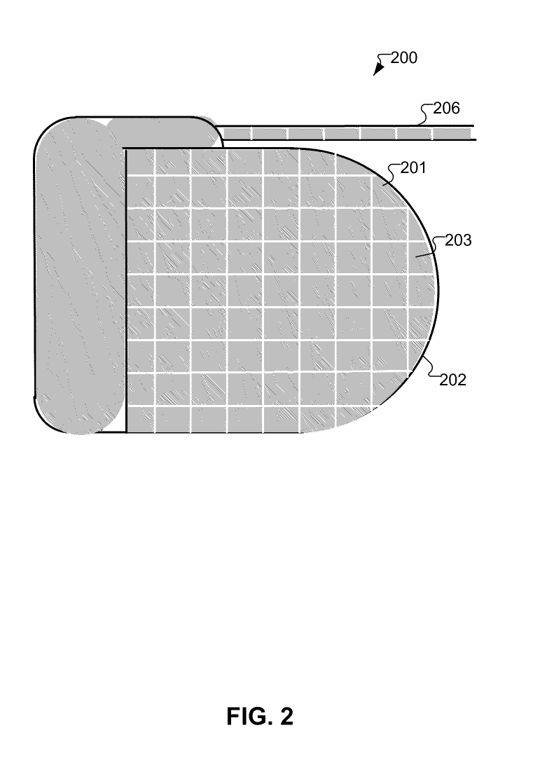

The coil housing 102 has a non-conductive outer surface 104, at least a portion of which is covered by the conductive coating 101 which forms a non-shielding pattern that minimizes interaction with the coil signal. The conductive coating 101 does not homogeneously cover the complete surface area of the coil housing 102, but rather is separated into smaller individual areas so that the negative influence on the inductive link is kept as small as possible, while at the same time, the area which is not coated shall be kept as small as possible so as to maximize the therapeutic benefits of the coating. For example, the non-shielding pattern of the conductive coating 101 may form a radial line pattern as shown in FIG. 1, or alternatively some other pattern such as a web or a mesh pattern (as in FIG. 2). The conductive coating 101 may be an antibiotic coating and/or a silver-based coating and/or a colloidal-based coating. Some embodiments may be limited by production processes and material properties (e.g. minimum effective thickness of non-conductive fragmentation lines) and efficacy of the conductive coating 101. The relative amount percentage of the surface of the coil housing 102 (and the dimension of areas) not covered by the conductive coating 101 and/or the size of the non-conductive fragmentation paths should be minimized to preserve good therapeutic properties. There may be further benefits to the use of a conductive coating 101 beyond the therapeutic antibiotic effect mentioned. For example:
to increase mechanical impact protection of the implantable device 100
to shield the implantable device 100 from ionizing radiation
   It may further be useful to pattern the conductive coating 101 as discussed above for such considerations.

The implantable device 100 may also contain a signal processing module 105 for processing the received coil signal. For example, in a cochlear implant system, the signal processing module 105 contains circuitry for developing electrode stimulation signals which are output through an attached electrode lead 106, the other end of which applies the stimulation signals to target nervous tissue. The conductive coating 101 may also cover some or all of the signal processing module 105 and/or the electrode lead 106 with or without the pattern used over the coil housing 102. For example, with a relatively long electrode lead 106 there may be a risk of RF-induced heating of the conductive coating 101, which can be mitigated by using a non-shielding (i.e. discontinuous or partitioned) pattern. There may be no conductive coating 101 over some elements of the implantable device 100 such as, for example, electrode ground contact 108.

FIG. 2 shows an example of another type of implantable device 200 having a mesh-patterned conductive coating 201 according to another embodiment of the present invention. In this embodiment, a single implant housing 202 made of a non-conductive ceramic material which contains the implanted coil 203 as well as the internal magnet and signal processing module (not shown). In this embodiment, the conductive coating 201 covers the entire implantable device 200 with the pattern extending over the implanted coil 203 and the electrode lead 206, with the remainder of the coating being unpatterned.

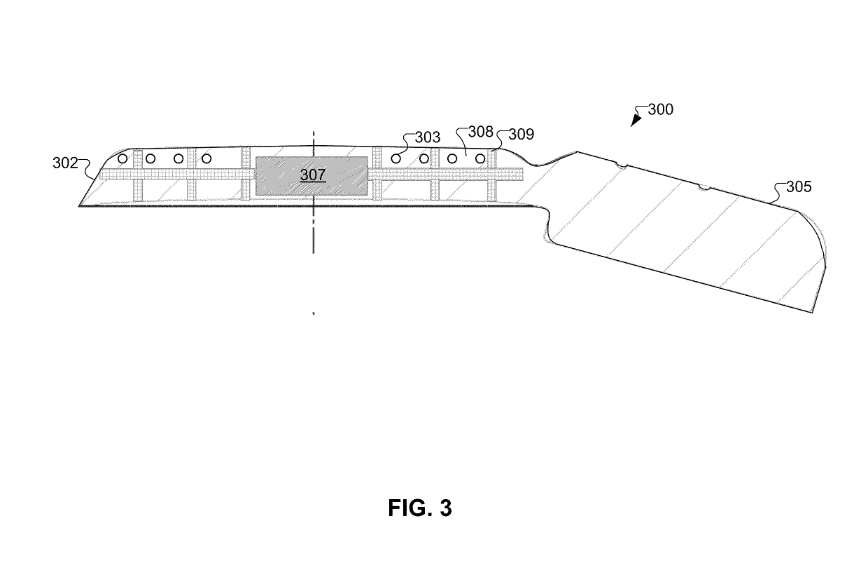

FIG. 3 shows a cross-sectional view of an implantable device 300 similar to the two-part device in FIG. 1, having a coil housing 302 and a separate signal processing module 305. Within the coil housing 302 are inductive link coils 303 for receiving a transcutaneous coil signal from an external transmitting coil. The coil housing 302 also contains an implanted magnet 307 that interacts with an external magnet to maintain the external magnet in a constant position adjacent to the implanted magnet.

The inductive link coils 303 are embedded in a low conductivity structure arranged in a non-shielding pattern of conductive containment material 308 (e.g., silicone impregnated with conductive material) which is divided by non-conductive separating structures 309, where the pattern minimizes interaction of the containment material 308 with the coil signal. In specific such embodiments, the non-shielding pattern may form a web, mesh, or radial line pattern. In the embodiment shown in FIG. 3, the non-conductive separating structures 309 separate individual link coils 303 from each other to minimize the shielding effect of the surrounding conductive containment material 308. The containment material 308 may include an antibiotic component and/or a silver-based component. It may be beneficial to implement non-conductive fragmentations need across the complete cross-section of the inductive link coils 303.

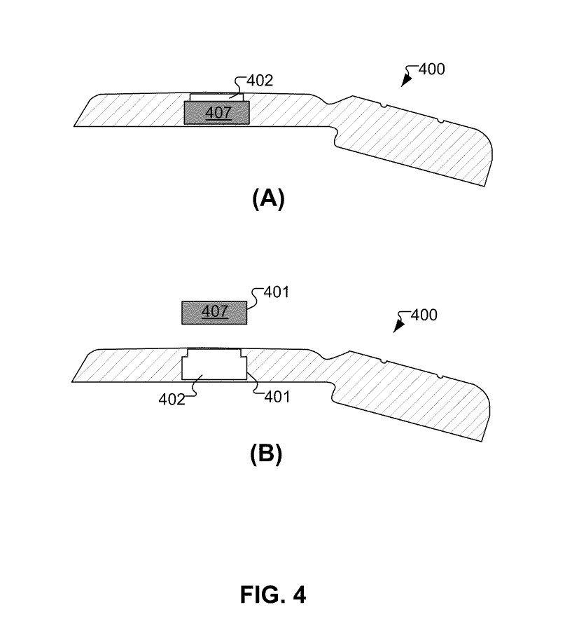

FIG. 4 A-B shows an implantable device 400 having a removable internal magnet 407 and using a therapeutic coating according to an embodiment of the present invention. The cylindrical internal magnet 407 is contained in a corresponding cylindrical magnet housing 402 and interacts with an external magnet to maintain the external magnet in a constant position adjacent to the implanted magnet 407. In one specific embodiment, the magnet housing 402 is in the form of a pocket of soft silicone material having an opening at the top through which the internal magnet 407 may be surgically removed when needed.

A therapeutic coating 401 covers the external surface of the implanted magnet 407 and the corresponding surfaces of the magnet housing 402 which engage the internal magnet 407. The therapeutic coating 401 provides of a therapeutic benefit such as preventing formation of a bio-film in the vicinity of the therapeutic coating, thereby avoiding infection. Specifically, the therapeutic coating 401 may include antibiotic coating and/or a silver-based coating. It may also be useful to provide a therapeutic coating 401 on any dummy parts (e.g., a non-metallic space holder replacing the internal magnet 407 during an MRI) and/or replacement magnets (inserted after the MRI).

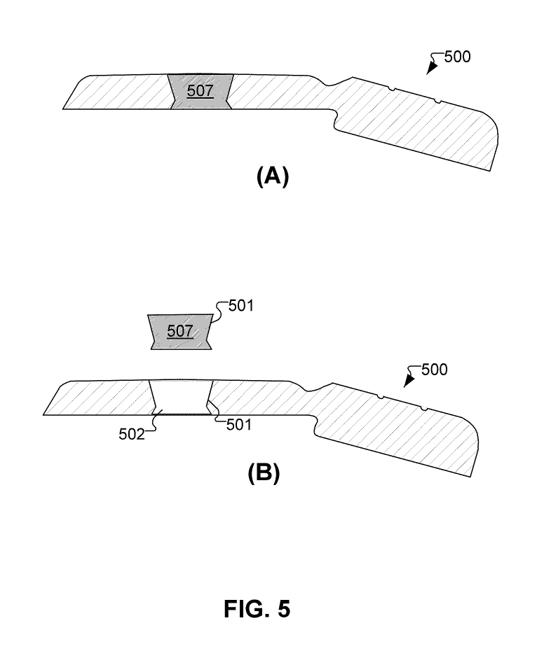

As with the conductive coatings discussed above, the therapeutic coating 401 may also be arranged in a non-uniform pattern. FIG. 5 A-B shows another implantable device 500 having a different shaped non-cylindrical removable internal magnet 507 and using a therapeutic coating 501 according to another embodiment of the present invention. In some specific embodiments, it may also be useful to physically seal the dead space between the magnet and the magnet housing and/or provide a tight fit between them that prevents micro-movements of the magnet relative to the magnet housing when the external coil is removed or placed over the implant, in order to further reduce the risk of bio-film growth in the magnet area.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable device comprising:
an implanted coil for receiving a transcutaneous coil signal from an external transmitting coil;
a coil housing containing the coil and having a non-conductive surface; and
a conductive coating covering at least a portion of the housing surface and formed in a non-shielding pattern that minimizes interaction with the coil signal.

2. An implantable device according to claim 1, wherein the non-shielding pattern forms a web pattern.

3. An implantable device according to claim 1, wherein the non-shielding pattern forms a mesh pattern.

4. An implantable device according to claim 1, wherein the non-shielding pattern forms a radial line pattern.

5. An implantable device according to claim 1, wherein the conductive coating is an antibiotic coating.

6. An implantable device according to claim 1, wherein the conductive coating is a silver-based coating.

7. An implantable device according to claim 1, wherein the conductive coating is a colloidal-based coating.

8. An implantable device according to claim 1, wherein the coil housing is formed of a ceramic material.

9. An implantable device according to claim 1, wherein the coil housing further contains a signal processing module for processing the received coil signal.

10. An implantable device according to claim 1, further comprising:
an electrode lead connected to the coil housing, wherein the conductive coating pattern further covers at least a portion of the electrode lead.

11. An implantable device according to claim 1, wherein the implantable device is an element in a cochlear implant system.

12. An implantable device comprising:
an implanted magnet that interacts with an external magnet to maintain the external magnet in a constant position adjacent to the implanted magnet;
a magnet housing containing the magnet; and
a therapeutic coating between at least a portion of the magnet and the magnet housing for delivery of a therapeutic benefit in the vicinity of the therapeutic coating.

13. An implantable device according to claim 11, wherein the therapeutic coating is an antibiotic coating and the therapeutic benefit includes an antibiotic effect.

14. An implantable device according to claim 11, wherein the therapeutic coating is a silver-based coating and the therapeutic benefit includes preventing formation of a biofilm in the vicinity of the therapeutic coating.

15. An implantable device according to claim 11, wherein the implanted magnet is a removable magnet.

16. An implantable device according to claim 11, wherein the magnet housing is formed of a ceramic material.

17. An implantable device according to claim 11, wherein the magnet housing further contains an implanted coil for receiving a transcutaneous coil signal from an external transmitting coil.

18. An implantable device according to claim 16, wherein the magnet housing further includes a signal processing module for processing the received coil signal.

19. An implantable device according to claim 11, wherein the implantable device is an element in a cochlear implant system.

20. An implantable device comprising:
an implanted coil for receiving a transcutaneous coil signal from an external transmitting coil; and
a coil housing containing the implanted coil embedded in a non-shielding pattern of conductive containment material divided by non-conductive separating structures, the pattern minimizing interaction of the containment material with the coil signal.

21. An implantable device according to claim 19, wherein the non-shielding pattern forms a web pattern.

22. An implantable device according to claim 19, wherein the non-shielding pattern forms a mesh pattern.

23. An implantable device according to claim 19, wherein the non-shielding pattern forms a radial line pattern.

24. An implantable device according to claim 19, wherein the containment material includes an antibiotic component.

25. An implantable device according to claim 19, wherein the containment material includes a silver-based component.

26. An implantable device according to claim 19, wherein the containment material includes a colloidal-based component.

27. An implantable device according to claim 19, wherein the coil housing is formed of a ceramic material.

28. An implantable device according to claim 19, wherein the coil housing further contains a signal processing module for processing the received coil signal.

29. An implantable device according to claim 19, wherein the implantable device is an element in a cochlear implant system.
